(19) United States
(12) Plant Patent Application Publication
(10) Pub. No.: US 2026/0283059 P1
(43) Pub. Date: Sep. 17, 2026

(54) Sweet Cherry Tree Named 'Final-bi 9.2'

(50) ;

(71) Applicant: Cerasina GmbH, Kressbronn (DE)

(72) Inventor: Peter STOPPEL, Kressbronn (DE)

(73) Assignee: Cerasina GmbH, Kressbronn (DE)

(21) Appl. No.: 19/563,187

(22) Filed: Mar. 11, 2026

(30) Foreign Application Priority Data
Mar. 12, 2025 (DE) KIA 00128

(51) Int. Cl. A01H 6/74; A01H 5/08

(52) U.S. Cl. CPC A01H 6/7445; A01H 5/08
USPC PLT/181

(57) ABSTRACT

A new and distinct variety of sweet cherry tree, referred to by its cultivar name, ‘Final-bi 9.2’, is described. The new plant variety forms fruit that is crisp and has a very good aroma. The fruit of the new variety is orange-red and yellow in color and has a teardrop shape.

Latin name of genus and species of plant claimed: Prunus avium (L.) L.

Variety denomination: ‘Final-bi 9.2’.

### CROSS REFERENCE TO RELATED APPLICATIONS

This application claims priority to Plant Breeders' Right Application No. KIA 00128, which was filed in Germany on Mar. 12, 2025, the contents of which are hereby incorporated by reference for all purposes.

### BACKGROUND OF THE INVENTION

The new plant variety of sweet cherry tree of the present invention was created by controlled breeding during spring flowering in 2001, in a controlled environment in Germany, by artificial pollination wherein two parents were crossed which previously had been studied in the hope that they would contribute the desired characteristics. The female parent (i.e., the seed parent) of the new plant variety was the ‘Späte von Wedler’ variety (not patented). The male parent (i.e., the pollen parent) of the new plant variety was the ‘Sweetheart’ variety (not patented).

The parentage of the new plant variety can be summarized as follows:
‘Späte von Wedler’ x ‘Sweetheart’

The seeds resulting from the above pollination were harvested in the fall of 2001. The seeds were sown and germinated, seedlings are raised in the spring of 2002, and flowering and fruiting occurred around 2006. These F1 population trees were observed, and selective study resulted in the identification of a single plant of the new plant variety.

The new plant variety has been found to undergo asexual propagation by cuttings and grafting. Asexual propagation techniques, such as cuttings, have shown that the characteristics of the new plant variety are homogeneous, stable, and strictly transmissible by such asexual propagation from one generation to another. Accordingly, the new plant variety undergoes asexual propagation in a true-to-type manner.

### SUMMARY OF THE INVENTION

It was found that the new variety of sweet cherry tree of the present invention possesses the following combination of characteristics:
(a) producing fruit that is crisp with a very good aroma, and
(b) exhibiting bi-color ripening period fruit.

The new plant variety can be readily distinguished from its ancestors. More specifically, the new plant variety produces fruit that ripens one week earlier and is more heart-shaped compared to fruit to the ‘Späte von Wedler’ variety (i.e., the seed parent). Additionally, the new plant variety is S4S4 S-alleles and produces fruit that ripens one week earlier and is orange-red and yellow colored compared to fruit of the ‘Sweetheart’ variety (i.e., the pollen parent), which is S3S4 S-alleles, and produces brown to red colored fruit which cracks faster than fruit of the new variety. Moreover, the new plant variety can be readily distinguished from non-parental related similar varieties. For example, the new plant variety produces fruit that is teardrop shape, has extensive orange-red coloration, and ripens earlier and is firmer compared to fruit of the ‘Prim-bi 3.2’ variety (U.S. Plant patent application Ser. No. 19/563,124), which produces fruit with a flatter shape and the red coloration is more speckled.

The new plant variety has been named the ‘Final-bi 9.2’ variety. The new plant variety has also been referred to as Breeder's Reference: ‘Stop 2011’.

### BRIEF DESCRIPTION OF THE PHOTOGRAPHS

The accompanying photographs show, as nearly true as it is reasonably possible to make the same in a color illustration of this character, a typical specimen of the new plant variety and fruit of the new plant variety.

FIG. 1 illustrates specimen of fruit growing on the new plant variety.

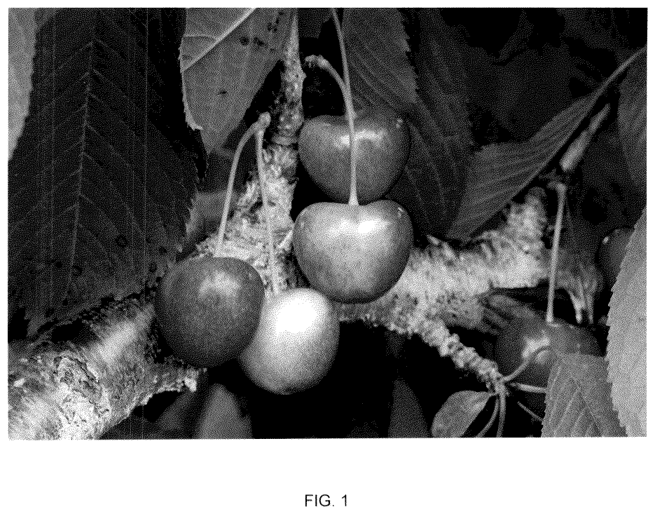

FIG. 2 illustrates specimen of the fruit of the new plant variety.

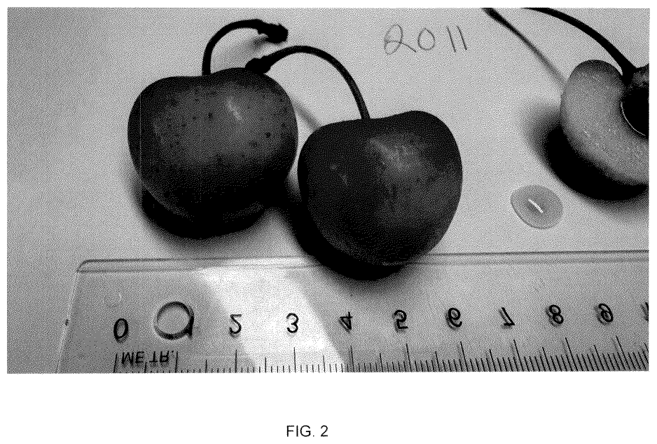

### DETAILED BOTANICAL DESCRIPTION

The chart used in the identification of colors is that of The Royal Horticultural Society (The R.H.S. Colour Chart, 2015 edition). The terminology which precedes reference to the chart has been added to indicate the corresponding color in more common terms. The R.H.S. Colour Chart designation used herein represents the closest color observed on the majority of the specified botanical features. The description is based on seven-years-old specimens of the new plant variety while growing on ‘Gisela 5’ rootstock in Kressbronn, Germany.
Botanical classification: Prunus avium (L.) L. cultivar ‘Final-bi 9.2’.
Commercial classification: Sweet Cherry Tree.
Tree:
   Vigor.—Medium.
   Height.—Approximately 3.0 m.
   Width.—Approximately 2.0 m.
   Growth habit.—Weak.
   Canopy density.—Medium.
   Form.—Spreading.
   Trunk diameter.—Approximately 10 cm above ground.
   Trunk surface texture.—Smooth with prominent lenticels.
   Lenticel length.—Typically 1.0 cm to 2.0 cm.
   Lenticel width.—Approximately 0.5 cm.
   Lenticel color.—Greyed-Orange Group 167A.
   Lenticel density.—Medium.
   Bark color.—Grey-Brown Group 199A.
Branches:
   Diameter.—Typically 0.5 cm to 1.5 cm.
   Surface.—Average, typical sweet cherry.
   Form.—Typical sweet cherry.
   Bark color.—Greyed-Orange Group 164A.
   Average angle.—Typically 60 degrees to 75 degrees.
   Lenticels length.—Approximately 1.0 mm.
   Lenticels width.—Approximately 1.0 mm.
   Lenticels shape.—Roundish.
   Lenticels color.—Greyed-Yellow Group 161B.
   Lenticels density.—Few, scattered.
Leaves:
   Length.—Typically 16.0 cm to 18.0 cm.
   Width.—Typically 8.0 cm to 9.0 cm.
   Form.—Narrow, pointed.
   Texture.—Leathery, crisp feeling.
   Thickness.—Medium.
   Base.—Trunkate.
   Apex.—Acute.
   Margin.—Serrations irregular.
   Color mature leaves upper surface.—Green Group 143A.
   Petiole shape.—Narrow.
   Petiole length.—Typically 4.0 cm to 5.0 cm.
   Petiole diameter.—Approximately 2.0 mm.
   Petiole color.—Adaxial Red-Purple Group 59A, abaxial Yellow-Green Group 149C.
   Venation type.—Pinnate.
   Veins color upper surface.—Yellow-Green Group 154D.
Flower buds:
   Length.—Approximately 10.0 mm.
   Width.—Approximately 1.5 mm.
   Color.—Reddish.
   Pedicel length.—Approximately 12.0 mm.
   Pedicel diameter.—Approximately 0.4 mm.
Flowers:
   Bloom timing.—Approximately Apr. 20, 2025.
   Blooming period.—Medium.
   Pollination requirement.—Self-fertile.
   Number of flowers per raceme.—Typically 3 to 7.
   Fragrance.—None.
   Diameter.—Approximately 3.0 cm.
   Petals number.—5.
   Petals length.—Approximately 10.0 mm.
   Petals width.—Approximately 12.0 mm.
   Petals shape.—Rounded medium-ovate.
   Petals margin.—Slightly repanded.
   Petals texture and appearance.—Soft texture.
   Petals color when opening.—Upper and lower surfaces White N999D.
   Petals color fully opened.—Upper and lower surfaces White N999D.
   Petal arrangement.—Free.
   Sepals shape.—Conical.
   Sepals margin.—Regular.
   Sepals length.—Typically 5.0 mm to 7.0 mm.
   Sepals width.—Approximately 5.0 mm.
   Sepals color.—Upper and lower surfaces are Green Group 143C.
   Stamens number per flower.—Approximately 37.
   Anthers shape.—Kidney.
   Anthers length.—Approximately 1.0 mm.
   Anthers color.—Yellow Group 13B.
   Pollen color.—Yellow Group 13B.
   Pollen amount.—Plentiful.
   Pistils.—Approximately 12.0 mm.
   Style length.—Approximately 5.0 mm.
   Style color.—Green Group 134C.
   Stigma shape.—Oval.
Fruit:
   Shape in ventral view.—Reniform.
   Diameter.—Typically between 28.0 mm to 30.0 mm.
   Fruit stalk length.—Approximately 55.0 mm.
   Fruit stalk thickness.—Approximately 2.0 mm.
   Skin color.—Red Group 41B.
   Skin ground color.—Yellow-Orange Group 20B.
   Skin thickness.—Firm.
   Skin texture.—Smooth.
   Skin tendency to crack.—Low tendency.
   Flesh color.—Yellow-Orange Group 16C.
   Firmness.—Very firm.
   Aroma.—Pleasant cherry flavor.
   Eating quality.—Good.
   Stone length.—Approximately 12.0 mm.
   Stone width.—Approximately 10.0 mm.
   Stone form.—Round.
   Stone color.—Greyed-Orange Group N168D.
   Stem length.—Approximately 55.0 mm.
   Stem width.—Approximately 2.0 mm.
   Stem color.—Green Group 137C.
Development:
   Time of beginning of flowering.—Medium.
   Time of beginning of fruit ripening.—Late to very late.
   Resistance to pathogens.—Cherry leaf roll virus (Nepovirus), Prune dwarf virus (Ilarvirus), Prunus necrotic ringspot virus (Ilarvirus), Tomato black ring virus (Nepovirus (subgroup B)), Tomato ringspot virus (Nepovirus lycopersici), Little cherry virus 1 (Velarivirus nanoavii), Little cherry virus 2 (Ampelovirus nanoavii).

The new ‘Final-bi 9.2’ variety has not been observed under all possible environmental conditions to date. Accordingly, it is possible that the phenotypic expression may vary somewhat with changes in light intensity and duration, cultural practices, and other environmental conditions.

I claim:

1. A new and distinct variety of sweet cherry tree named ‘Final-bi 9.2’ substantially as illustrated and described herein.
